(19) United States
(12) Patent Application Publication
(10) Pub. No.: US 2022/0273750 A1
(43) Pub. Date: Sep. 1, 2022

(54) PERFORMANCE ENHANCING BEVERAGE

(71) Applicant: Mel Chinn, Ferguson, MO (US)

(72) Inventor: Mel Chinn, Ferguson, MO (US)

(21) Appl. No.: 17/589,034

(22) Filed: Jan. 31, 2022

Related U.S. Application Data
(60) Provisional application No. 63/154,111, filed on Feb. 26, 2021.

(51) Int. Cl. A61K 36/752; A61K 31/7048; A61K 33/00; A61K 33/06; A61K 33/34; A61K 33/26; A61K 33/30; A61K 31/593; A61K 31/198; A61K 9/00; A23L 2/02; A23L 2/52; A23L 33/105; A23L 33/16; A23L 33/155; A23L 33/175; A23L 33/00

(52) U.S. Cl. CPC A61K 36/752; A61K 31/7048; A61K 33/00; A61K 33/06; A61K 33/34; A61K 33/26; A61K 33/30; A61K 31/593; A61K 31/198; A61K 9/0095; A23L 2/02; A23L 2/52; A23L 33/105; A23L 33/16; A23L 33/155; A23L 33/175; A23L 33/40; A23V 2002/00

(57) ABSTRACT

Performance enhancing beverages, particularly sexual performance, comprising an alkaline consumable liquid and one or more sources of flavanones and methods of producing the same. Methods of administering and/or consuming beverages to enhance sexual performance are also described. Once administered and/or consumed, an individual's blood flow is increased, which can enhance sexual performance during intercourse. If the individual is a human male, consuming an effective amount of the beverage may cause an erection and/or improve erectile function.

### CROSS-REFERENCE TO RELATED APPLICATIONS

The present application claims the priority benefit of U.S. Provisional Patent Application Ser. No. 63/154,111, filed Feb. 26, 2021, entitled PERFORMANCE ENHANCING BEVERAGE, incorporated by reference in its entirety herein.

### BACKGROUND OF THE DISCLOSURE

### Field

The present disclosure relates to a beverage for enhancing performance, particularly sexual performance, and to methods of producing the same. Once administered and/or consumed, the beverage increases an individual's blood flow, which enhances sexual performance during intercourse.

### Description of Related Art

Typical erectile dysfunction drugs, such as Viagra, Cialis, Levitra, are selective inhibitors of cyclic guanosine monophosphate (cGMP)-specific phosphodiesterase type 5 (PDE5). The inhibition of PDES enhances erectile function by increasing the amount of cGMP, which, when released, causes smooth muscle relaxation and increased blood flow into the corpus cavernosum. However, an individual can experience unwanted or severe side effects when taking PDES inhibitors alone or in combination with other medicines.

Therefore, there is a need for an all-natural beverage that can improve blood flow and enhance an individual's sexual performance without causing unwanted side effects.

### SUMMARY OF THE DISCLOSURE

The present disclosure is broadly concerned with performance enhancing beverages. Embodiments include methods of enhancing sexual performance, such as by administering to an individual an effective amount of a performance enhancing beverage, the performance enhancing beverage comprising (consisting essentially of even consisting of): from about 80% to about 99% by weight of a consumable liquid having a pH of 7 or greater; and from about 1% to about 20% by weight of one or more sources of flavanones, wherein each % by weight is based upon the total weight of the beverage taken as 100% by weight, wherein the performance enhancing beverage is effective for, inter alia, increasing blood flow and/or enhancing sexual performance in the individual.

Also disclosed are methods of producing a performance enhancing beverage, the method comprising mixing a consumable liquid having a pH of 7 or greater, with one or more sources of flavanones to form a mixture, wherein the one or more sources of flavanones is a liquid extract and/or pieces of a fruit produced by a Citrus plant.

Also disclosed are performance enhancing beverages which comprise (consist essentially or even consist of) from about 80% to about 99% by weight of a consumable liquid having a pH of 7 or greater, from about 1% to about 20% by weight of a non-microencapsulated liquid extract and/or pieces from an organic lemon or lime; and an ingredient selected from the group consisting of potassium, magnesium, calcium, sodium, copper, iron, and zinc, Vitamin D3, L-arginine, and mixtures thereof, wherein the performance enhancing beverage is effective for increasing blood flow and/or enhancing sexual performance in the individual and is free from sildenafil citrate, tadalafil, vardenafil, avanafil, or mixtures thereof.

### DETAILED DESCRIPTION

The present disclosure is broadly concerned with performance enhancing beverages and methods of producing the same. Also described are methods of enhancing sexual performance.

### Performance Enhancing Beverages

In one or more embodiments, the performance enhancing beverages comprise a consumable liquid and one or more sources of flavanones.

### 1. Ingredients

In most embodiments, the beverages comprise an alkaline consumable liquid having a pH of 7 or greater. In preferred embodiments, the consumable liquid has a pH range of about 7.3 to about 7.5. Advantageously, at this pH range, the consumable liquid matches the pH range of the individual's body, allowing the body to maintain its preferred alkaline environment. In particular, because the optimum pH range of the human body is about 7.4, in certain preferred embodiments, the pH of the consumable liquid is about 7.3 to about 7.4 (preferably about 7.4).

The consumable liquid may be any liquid suitable to be consumed by and/or administered to a human, such as, but not limited to water, tea, sports (electrolyte) drinks, caffeinated drinks, but is preferably water. If the consumable liquid is water, the water may be purified water, tap water, mineral water, spring water, distilled water, well water, sparkling (i.e., carbonated) water, non-carbonated water, infused water, flavored water, and is preferably purified water. The purified water may be any water mechanically filtered or processed to remove impurities (e.g., reverse osmosis, ozonation, ultra-violet light exposure), preferably an ultra-purified Spring water with a pH of from about 7.3 to about 7.4. In other embodiments, the water may further include a number of optional ingredients, including but not limited to, minerals (e.g., calcium chloride, magnesium chloride, and potassium bicarbonate), salts, electrolytes, or combinations thereof. The beverages comprise about 80% to about 99%, preferably about 75% to about 97%, more preferably about 88% to about 95%, most preferably about 91% of the consumable liquid, wherein the % by weight is based upon the total weight of the beverages each taken as 100% by weight.

In most embodiments, the beverages comprise one or more sources of flavanones, which may be in a dry or liquid form, preferably liquid form. Flavonoids include a class of aromatic secondary metabolites that are commonly found in plants and provide health benefits to an individual, such as antioxidant effects. The liquid extract from fruits produced by a Citrus plant is a rich source of flavonoids, particularly flavanones. In preferred embodiments, the one or more sources of flavanones may be the liquid extract from a citrus fruit, such as lemons, limes, oranges, mandarin oranges, grapefruits, clementines, tangerines, citrons, pomelos, preferably lemons or limes. If the one or more sources of flavanones is the liquid extract from a lemon, the variety of lemon may be, but is not limited to, Citrus limon, Avalon lemon (Citrus x limon ‘Avon’), Bearss lemon (Citrus x limon ‘Bearss’), Eureka lemon (Citrus x limon ‘Eureka’), Harvey lemon (Citrus x limon ‘Harvey’), Lisbon lemon (Citrus x limon ‘Lisbon’), Meyer lemon (Citrus x meyeri), and Ponderosa Lemon (Citrus x pyriformis). If the one or more sources of flavanones is the liquid extract from a lime, the variety of lime may be, but is not limited to, Bearss lime (Citrus latifolia), Limequats (Citrofortunella x floridana), Key lime (Citrus aurantiifolia), and Rangpur lime (Citrus x limonia). The one or more sources of flavanones may be organic or non-organic, preferably organic. The one or more sources of flavanones may provide flavanones, flavanone aglycones, flavanone glycosides (e.g., rutinose and neohesperidose glycoside), or mixtures thereof. In preferred embodiments, the one or more sources of flavanones contains flavanone glycosides, such as hesperidin, eriocitrin, narirutin, didymin, naringin, preferably hesperidin and eriocitrin.

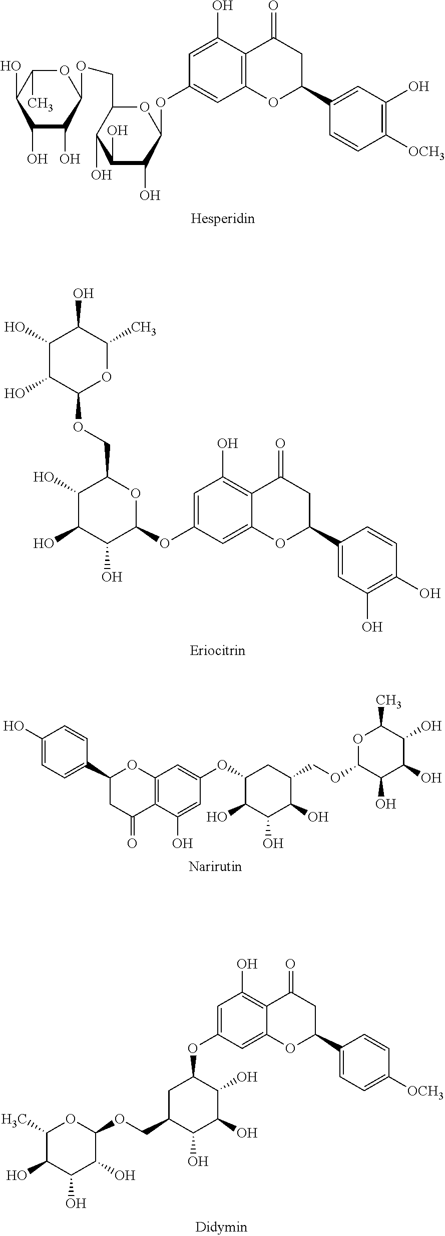

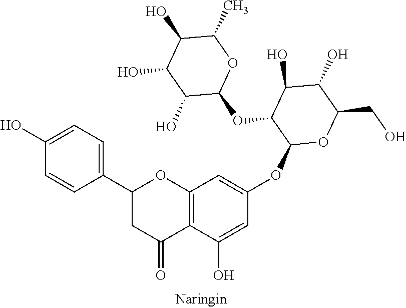 [SMILES: C[C@@H]1O[C@@H](O[C@H]2[C@H](Oc3cc(O)c4c(c3)OC(c3ccc(O)cc3)CC4=O)O[C@H](CO)[C@@H](O)[C@@H]2O)[C@H](O)[C@H](O)[C@H]1O]

The beverages comprise about 1% to about 20%, preferably about 3% to about 20%, more preferably about 5% to about 12%, most preferably about 9% of the one or more sources of flavanones, wherein the % by weight is based upon the total weight of the beverages each taken as 100% by weight.

In one or more embodiments, the weight ratio of the consumable liquid and the one or more sources of flavanones is about 99:1 to about 4:1, preferably about 30:1 to about 5:1, more preferably about 20:1 to about 10:1, most preferably about 10:1.

In one or more embodiments, the beverage includes not just the juice as the source of flavonones, but also pieces of the fruit itself (e.g., lemon and/or lime wedges) submerged in the ultra-purified Spring water (pH 7.3-7.4) enhanced with electrolytes and minerals. Thus, it will be appreciated that the weight percents described above relate primarily to the amount of juice or extract in the beverage, and may not account for the bulk pith, flesh, and/or rind of the fruit. On a volume basis, up to 30% of the volume of the beverage may include the source of flavonones, particularly in the case of pieces or fragments of the fruit being submerged in the beverage.

### 2. Other Ingredients

It will be appreciated that a number of other optional ingredients can be included in the beverages as well. Typical optional ingredients include minerals and electrolytes (e.g., potassium, magnesium, calcium, sodium, chloride salts), pro-oxidants (e.g., copper and iron), antioxidant nutrients (e.g., copper, zinc, selenium, vitamin D3), amino acids (e.g., L-arginine), and the like. One of ordinary skill in the art will be able to select one or more suitable minerals, electrolytes, pro-oxidants, antioxidant nutrients, or combinations thereof for beverages according to this disclosure. In one or more embodiments, these additional ingredients are included in the beverage in amounts suitable to provided recommended daily dosage of each ingredient within the table below (for enhanced performance). It will be appreciated that the amounts of each ingredient in the beverage can be increased or decreased depending upon the recommended serving size of each beverage and number of servings to be consumed daily to achieve the recommended daily dosages listed below. For example, one serving of the beverage may contain half of the recommended daily dosage mentioned below, with the recommended serving being two beverages per day. Likewise, one serving of the beverage may contain a third of the recommended daily dosage of the ingredients mentioned below, with the with the recommended serving being three beverages per day.

| | Total | |
|---|---|---|
| Ingredient | Daily Dosage | |
| Potassium | 1700 | mg |
| Magnesium | 200-210 | mg |
| Calcium | 500 | mg |
| Sodium | 355 | mg |
| Copper | 700 | μg |
| Iron | 4 | mg |
| Zinc | 5.5 | mg |
| Vitamin D3 | 12-50 | μg |
| L-arginine | 2.5-5 | g |

Advantageously, when consumed, the beverages increase an individual's blood flow, which can cause enhanced sexual performance during intercourse. In addition to the foregoing, if administered to a human, the beverages also help maintain an alkaline environment in the human body, prevent dehydration, increase the potency of vitamin C, lower blood sugar, help reduce heart disease, assist in weight loss, reduce inflammation, and the like.

It will be appreciated that the beverages do not comprise (i.e., are essentially free of) a phosphodiesterase-5 enzyme inhibitor, such as sildenafil citrate, tadalafil, vardenafil, and avanafil, as well as other undesirable ingredients such as artificial sweeteners, aspartame, sucralose, saccharin, neotame, and acesulfame potassium, high fructose corn syrup, erythritol or maltodextrin, artificial dyes and coloring agents (e.g., Red 3 or 40, Blue 1 or 2, Yellow 5 or 6, and the like). If sweetening is desired, it can be provided by more natural sweeteners such as honey, raw sugar, sucrose, stevia, and the like. As used herein, “essentially free” means that the ingredient is not intentionally added to the beverage but may be present in trace or incidental amounts (e.g., less than 1% by weight, preferably less than 0.5% by weight, and preferably less than 0.1% by weight).

### Method of Producing the Performance Enhancing Beverages

The present disclosure also provides methods of producing the performance enhancing beverages. The beverages are generally produced by mixing the consumable liquid with the one or more sources of flavanones. In preferred embodiments, under ambient conditions, the one or more sources of flavanones is added to the consumable liquid and mixed until homogenous using a suitable mixer, such as a stirring spoon. As used herein, the phrase “ambient conditions” refers to the general environmental conditions of the area in which the method is being performed, with no external heat or cooling provided. If the one or more sources of flavanones is the liquid extract from a fruit produced by a Citrus plant, the fruit is preferably first sliced into wedges before the liquid extract is added to the consumable liquid. It will be appreciated that, in all embodiments, the one or more sources of flavanones are not microencapsulated when added to the consumable liquid. In some embodiments, optional ingredients, such as minerals, electrolytes, pro-oxidants, and antioxidant nutrients, may be added to the consumable liquid and/or the one or more sources of flavanones before each are mixed together. In other embodiments, optional ingredients are added to the beverage after it is formed. After the beverage is prepared, the beverage may be administered by any means, preferably by ingestion.

### Method for Enhancing Sexual Performance

Once the beverages are produced according to the methods described above, the beverages may be administered to increase blood flow. In most embodiments, an effective amount of the beverage is administered to and/or consumed by an individual, preferably a human, more preferably a human male, before intercourse. The beverage is preferably consumed by ingestion, but any suitable means can also be used. As used herein, an “effective amount” of the beverage refers to an amount capable of increasing blood flow, enhancing sexual performance, and/or improving erectile function in an individual. In preferred embodiments, about 10 fluid ounces to about 30 fluid ounces, preferably 20 fluid ounces of the beverage may be ingested before intercourse. When an effective amount of the beverage is administered, it will be appreciated that the beverage increases the individual's blood flow, which can enhance the individual's sexual performance. In certain preferred embodiments, if the individual is a human male, consuming an effective amount of the beverage may cause an erection and/or improve erectile function. If desired, to further improve erectile function, an individual may ingest about 1 gallon to about 2 gallons, preferably about 1 gallon of the beverage per day.

It will also be appreciated that the beverage may be consumed within a certain time before intercourse. In some embodiments, an effective amount of the beverage may be consumed immediately before intercourse. In other embodiments, an effective amount of the beverage may be consumed about 60 minutes to about 10 minutes, preferably about 40 minutes to about 30 minutes, more preferably 20 minutes before intercourse. In most preferred embodiments, 20 fluid ounces of the beverage is ingested 20 minutes before intercourse.

Additional advantages of the various embodiments of the disclosure will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the disclosure. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration, and nothing therein should be taken as a limitation upon the overall scope of the invention.

### Example 1

The beverage was prepared using 10 fluid ounces of ultra-purified Spring water (pH 7.3-7.4) enhanced with electrolytes and minerals, 1 tablespoon of organic lemon juice, and 1 tablespoon of organic lime juice. To produce the lemon juice and lime juice, an organic lemon and an organic lime were cut into slices approximately ¼ of each fruit, and squeezed until a tablespoon of juice was extracted from each. The extracted lemon juice and the extracted lime juice was then mixed with the ultra-purified Spring water (pH 7.3-7.4) enhanced with electrolytes and minerals along with the sliced fruit pieces to form the beverage (“12 Springs and 70 Palm Trees”).

### Example 2

Another variation of the beverage was prepared using 10 fluid ounces of ultra-purified Spring water (pH 7.3-7.4) enhanced with electrolytes and minerals and 2 tablespoons of organic lemon juice. To produce the juice, an organic lemon was cut into slices approximately ¼ of each fruit and squeezed until two tablespoons of juice was extracted. The extracted juice was then mixed with the ultra-purified Spring water (pH 7.3-7.4) enhanced with electrolytes and minerals along with the sliced fruit pieces to form the beverage (“12 Springs and 70 Palm Trees”).

### Example 3

Another variation of the beverage was prepared using 10 fluid ounces of ultra-purified Spring water (pH 7.3-7.4) enhanced with electrolytes and minerals and 2 tablespoons of organic lime juice. To produce the juice, an organic lime was cut into slices approximately ¼ of each fruit and squeezed until two tablespoons of juice was extracted. The extracted juice was then mixed with the ultra-purified Spring water (pH 7.3-7.4) enhanced with electrolytes and minerals along with the sliced fruit pieces to form the beverage (“12 Springs and 70 Palm Trees”).

### Example 4

Another variation of the beverage was prepared using 10 fluid ounces of ultra-purified Spring water (pH 7.3-7.4) enhanced with electrolytes and minerals, 1 tablespoon of non-organic lemon juice, and 1 tablespoon of non-organic lime juice. To produce the lemon juice and the lime juice, a non-organic lemon and a non-organic lime were cut into slices approximately ¼ of each fruit and squeezed until a tablespoon of juice was extracted from each. The extracted juice was then mixed with the ultra-purified Spring water (pH 7.3-7.4) enhanced with electrolytes and minerals along with the sliced fruit pieces to form the beverage (“12 Springs and 70 Palm Trees”).

### Example 5

Another variation of the beverage was prepared using 10 fluid ounces of ultra-purified Spring water (pH 7.3-7.4) enhanced with electrolytes and minerals and 2 tablespoons of non-organic lemon juice. To produce the juice, a non-organic lemon was cut into slices approximately ¼ of each fruit and squeezed until two tablespoons of juice was extracted. The extracted juice was then mixed with the ultra-purified Spring water (pH 7.3-7.4) enhanced with electrolytes and minerals along with the sliced fruit pieces to form the beverage (“12 Springs and 70 Palm Trees”).

### Example 6

Another variation of the beverage was prepared using 10 fluid ounces of ultra-purified Spring water (pH 7.3-7.4) enhanced with electrolytes and minerals and 2 tablespoons of non-organic lime juice. To produce the juice, a non-organic lime was cut into slices approximately ¼ of each fruit and squeezed until two tablespoons of juice was extracted. The extracted juice was then mixed with the ultra-purified Spring water (pH 7.3-7.4) enhanced with electrolytes and minerals along with the sliced fruit pieces to form the beverage (“12 Springs and 70 Palm Trees”).

I claim:

1. A method of increasing blood flow and/or enhancing sexual performance in an individual, the method comprising:
administering to the individual an effective amount of a performance enhancing beverage, the performance enhancing beverage comprising:
about 80% to about 99% by weight of a consumable liquid having a pH of 7 or greater; and
about 1% to about 20% by weight of one or more sources of flavanones,
wherein each % by weight is based upon the total weight of the beverage taken as 100% by weight,
wherein the performance enhancing beverage is effective for increasing blood flow and/or enhancing sexual performance in the individual.

2. The method of claim 1, wherein the effective amount is about 10 fluid ounces to about 30 fluid ounces of the beverage.

3. The method of claim 1, wherein the individual is a human male.

4. The method of claim 1, wherein the consumable liquid has a pH of from about 7 to about 7.5.

5. The method of claim 4, wherein the consumable liquid has a pH of from about 7.3 to about 7.4.

6. The method of claim 1, wherein the one or more sources of flavanones is a liquid extract from a fruit produced by a Citrus plant.

7. The method of claim 6, wherein the one or more sources of flavonones further comprises pieces or fragments of said fruit dispersed in said consumable liquid.

8. The method of claim 6, wherein the fruit is selected from the group consisting of lemons, limes, oranges, mandarin oranges, grapefruits, clementines, tangerine, citrons, pomelos, and mixtures thereof.

9. The method of claim 8, wherein the fruit is an organic lemon and/or an organic lime.

10. The method of claim 1, wherein the flavanone is a flavanone glycoside.

11. The method of claim 10, wherein the flavanone glycoside is hesperidin or eriocitrin.

12. The method of claim 1, wherein the beverage further comprises an ingredient selected from the group consisting of potassium, magnesium, calcium, sodium, copper, iron, and zinc, Vitamin D3, L-arginine, and mixtures thereof.

13. The method of claim 1, wherein the beverage is free from sildenafil citrate, tadalafil, vardenafil, avanafil, or mixtures thereof.

14. A method of producing a performance enhancing beverage, the method comprising mixing a consumable liquid having a pH of 7 or greater, with one or more sources of flavanones to form a mixture, wherein the one or more sources of flavanones is a liquid extract and/or pieces of a fruit produced by a Citrus plant.

15. The method of claim 14, wherein the method further comprises adding to the mixture an ingredient selected from the group consisting of potassium, magnesium, calcium, sodium, copper, iron, and zinc, Vitamin D3, L-arginine, and mixtures thereof.

16. The method of claim 14, wherein the method further comprises cutting the fruit into pieces before the mixing step and extracting juice from said pieces.

17. The method of claim 16, further comprising adding said pieces are to the consumable liquid.

18. The method of claim 14, wherein the one or more sources of flavanones is not microencapsulated.

19. A performance enhancing beverage produced according to the method of claim 14.

20. A performance enhancing beverage comprising:
about 80% to about 99% by weight of a consumable liquid having a pH of 7 or greater;
about 1% to about 20% by weight of a non-microencapsulated liquid extract and/or pieces from an organic lemon or lime; and
an ingredient selected from the group consisting of potassium, magnesium, calcium, sodium, copper, iron, and zinc, Vitamin D3, L-arginine, and mixtures thereof,
wherein each % by weight is based upon the total weight of the beverage taken as 100% by weight,
wherein the performance enhancing beverage is effective for increasing blood flow and/or enhancing sexual performance in the individual and is free from sildenafil citrate, tadalafil, vardenafil, avanafil, or mixtures thereof.
